(19) United States
(12) Patent Application Publication
(10) Pub. No.: US 2012/0202250 A1
(43) Pub. Date: Aug. 9, 2012

(54) Method for Exponential Amplification of RNA Using Thermostable RNA-dependent RNA Polymerase

(75) Inventor: Jacques Rohayem, Dresden (DE)

(73) Assignee: RiboxX GmbH, Radebeul (DE)

(21) Appl. No.: 13/502,402

(22) PCT Filed: Oct. 21, 2010
(86) PCT No.: PCT/EP2010/065904

§ 371 Date: Apr. 17, 2012

(30) Foreign Application Priority Data
Oct. 21, 2009 (EP) 09173697.5
Nov. 10, 2009 (EP) 09175558.7

(51) Int. Cl. C12P 19/34; B01J 19/12

(52) U.S. Cl. 435/91.21; 204/157.68

(57) ABSTRACT

The present invention relates to a method for exponential amplification of RNA in vitro by using a thermostable RNA-dependent RNA polymerase (RdRp) of a sapovirus or norovirus.

The present invention relates to a method for exponential amplification of RNA in vitro by using a thermostable RNA-dependent RNA polymerase (RdRp) of a sapovirus or norovirus.

Ever since the provision of the polymerase chain reaction (PCR; cf. EP 0 200 326 B1), one major breakthrough in the development of modern DNA amplification techniques was the use of thermostable DNA polymerases such as Taq polymerase (see U.S. Pat. No. 4,889,818).

In comparison to DNA amplification by PCR, existing RNA amplification methods suffer from several drawbacks: protocols for mRNA amplification using T7 polymerase (SMART™ mRNA Amplification Kit User Manual, Clontech Laboratories, Inc., 28 Apr. 2008; U.S. Pat. No. 5,962,271, U.S. Pat. No. 5,962,272) include complex and time consuming enzymatic steps:

1) reverse transcription step of producing a double-stranded cDNA from the RNA which is to be amplified. This occurs usually with a primer-dependent RNA-dependent DNA-polymerase, e.g. from Avian Myeloblastosis Virus (AMV) or Moloney Murine Leukemia Virus (MuLV).
2) The produced double-stranded cDNA is then used as a template to synthesize RNA by the T7 polymerase. The T7-Polymerase is a primer-dependent DNA-dependent RNA-Polymerase and requires a T7 specific promoter sequence within the primer sequence for initiation of polymerisation.
Amplification of RNA by the T7 Polymerase occurs in a linear fashion and is performed usually at 37° C. The T7 Poymerase does not tolerate temperatures higher than 50° C. for its activity.

Another enzyme which has been suggested for RNA amplification is Qβ replicase (see WO 02/092774 A2). Qβ replicase is a RNA-dependent RNA-polymerase that needs a primer having a sequence-specific recognition site for initiation of RNA polymerisation. Protocols of this type only achieve linear RNA amplification and are performed usually at 37° C. The Qβ replicase does not tolerate temperatures higher than 50° C. for its activity.

Furthermore, RNA amplification using polymerases from bacteriophages Phi-6 to Phi-14 (cf. WO 01/46396 A1) requires the presence of a specific promoter sequence. Phi-6 to Phi-14 enzymes are RNA-dependent RNA-polymerases. Also in this case only linear amplification has been achieved with such enzymes, occurring at 37° C. The Phi-6 to Phi-14 enzymes do not tolerate temperatures higher than 50° C. for its activity.

WO 2007/12329 A2 discloses a method for preparing and labelling RNA using a (RNA-dependent RNA-polymerase) RdRp of the family of Caliciviridae. The authors show successful de novo RNA synthesis from single-stranded RNA (ssRNA) templates in the presence or absence of a RNA-synthesis initiating oligonucleotide (oligoprimer with a length less than 10 nt) and also envisage repeated cycling of RNA synthesis and denaturation of the double-stranded RNA (dsRNA) products. Exponential RNA amplification is not shown in WO 2007/12329 A2, and the reaction is described to occur at 20° C. to 40° C.

The technical problem underlying the present invention is to achieve exponential amplification of RNA by implementing a novel method for large-scale enzymatic synthesis of RNA. This novel method makes use of a thermostable RNA-dependent RNA polymerase, allowing exponential amplification of RNA starting from a single RNA template.

The solution to the above technical problem is provided by the embodiments of the present invention as characterised in the claims.

The inventors have surprisingly found that exponential amplification of RNA templates is feasible by employing a sapovirus or norovirus RdRp which is essentially stable and active at temperatures of up to about 85° C.

Therefore, the present invention provides a method for exponential amplification of RNA in vitro comprising the steps of:

(a) incubating single-stranded RNA (ssRNA) with a RNA-dependent RNA polymerase (RdRp) of a sapovirus or norovirus, optionally in the presence of an RNA-synthesis initiating oligonucleotide (oligoprimer), under conditions such that the RdRp polymerises a strand complementary to the ssRNA, optionally by elongating said oligoprimer hybridized to said ssRNA, to form double-stranded RNA (dsRNA);
(b) incubating the reaction mixture obtained in step (a) at a temperature of at most 85° C., preferably 65° C. to 85° C., such that the duplex of the dsRNA is separated into ssRNA;
(c) repeating steps (a) and (b) n times;
(d) performing a final incubation step (a) to form final dsRNA; and, optionally,
(e) recovering the final dsRNA;
wherein
n is at least 3, preferably 5 to 40, particularly preferred 20;
and the sequence and/or length of the ssRNA is selected such that the dsRNA formed in step (b) is separated into ssRNA at a temperature of at most 85° C., preferably at a temperature of from 65° C. to 85° C.

In case of amplifying polyadenylated RNA (in particular mRNA) an RNA-synthesis initiating oligonucleotide (oligo- or polyU primer) is required. Correspondingly, amplification of polyguanylated and polyuridylated RNA requires an oligoC (or polyC) and oligoA (or polyA), respectively, primer. In the case of polycytidylated templates RNA synthesis can either be initiated by using an oligoG (or polyG) primer or it can be initiated de novo (i.e. in the absence of an RNA-synthesis initiating oligonucleotide) using GTP in surplus (preferably, 2× 3×, 4' or 5× more) over ATP, UTP and CTP, respectively.

Although not essential, the sapovirus RdRp may lose some of its activity during repeated heating steps, especially at temperatures above 80° C. Thus, in case of n≧5, further RdRp may be added between step (b) and (a) at every 3^{rd} to 5^{th} cycle of step (c).

Preferably, the sapovirus RdRp is an RdRp of the sapovirus strain pJG-Sap01 (GenBank Acc. No. AY694184). A norovirus RdRp useful in the present invention is preferably an RdRp of the norovirus strain NuCV/NL/Dresden174/1997/GE (GenBank Acc. No. AY741811). Sapovirus or norovirus RdRps for use in the present invention may be prepared by recombinant expression methods known in the art (see WO 2007/12329 A2). In this context, it is also contemplated to use enzymes having a “tag” that facilitates recombinant expression and/or purification. A preferred tag is a His-tag which may be present either at the C- or N-terminus of the respective recombinant enzyme.

Preferably, the sapovirus RdRp has an amino acid sequence according to SEQ ID NO: 1, SEQ ID NO: 2 or SEQ ID NO: 3:

SEQ ID NO: 1:
MKDEFQNKGLPVVKSGLDVGGMPTGTRYHRSPAWPEEQPGETHAPAPFG
AGDKRYTFSQTEMLVNGLKPYTEPTAGVPPQLLSRAVTHVRSYIETIIG
THRSPVLTYHQACELLERTTSCGPFVQGLKGDYWDEEQQQYTGVLANHL
EQAWDKANKGIAPRNAYKLALKDELRPIEKNKAGKRRLLWGCDAATTLI
ATAAFKAVATRLQVVTPMTPVAVGINMDSVQMQVMNDSLKGGVLYCLDY
SKWDSTQNPAVTAASLAILERFAEPHPIVSCAIEALSSPAEGYVNDIKF
VTRGGLPSGMPFTSVVNSINHMIYVAAAILQAYESHNVPYTGNVFQVET
VHTYGDDCMYSVCPATASIPHAVLANLTSYGLKPTAADKSDAIKPTNTP
VFLKRTFTQTFHGVRALLDITSITRQFYWLKANRTSDPSSPPAFDRQAR
SAQLRNALAYASQHGPVVFDTVRQIAIKTAQGEGLVLVNTNYDQALATY
NAWFIGGTVPDPVGHTEGTHKIVFEME
SEQ ID NO: 2:
MKDEFQWKGLPVVKSGLDVGGMPTGTRYHRSPAWPEEQPGETHAPAPFG
AGDKRYTFSQTEMLVNGLKPYTEPTAGVPPQLLSRAVTHVRSYIETIIG
THRSPVLTYHQACELLERTTSCGPFVQGLKGDYWDEEQQQYTGVLANHL
EQAWDKANKGIAPRNAYKLALKDELRPIEKNKAGKRRLLWGCDAATTLI
ATAAFKAVATRLQVVTPMTPVAVGINMDSVQMQVMNDSLKGGVLYCLDY
SKWDSTQNPAVTAASLAILERFAEPHPIVSCAIEALSSPAEGYVNDIKF
VTRGGLPSGMPFTSVVNSINHMIYVAAAILQAYESHNVPYTHNVGQVET
VHTYGDDCMYSVCPATASIFHAVLANLTSYGLKPTAADKSDAIKPTNTP
VFLKRTFTQTPHGVRADDDITSITRQFYWLKANRTSDPSHPPAFDRQAR
SAQLENALAYASQHGPVVFDTVRQIAIKTAQGHGLVLVNTNYDQALATY
NAWFIGGTVPDFVGHTEGTHKIVFEMEHHHHHH
SEQ ID NO: 3
MKHHHHHHDEFQWKGLPVVKSGLDVGGMPTGTRYHRSPAWPEEQPGETH
APAPFGAGDKRYTFSQTEMLVNGLKPYTEPTAGVPPQLLSRAVTHVRSY
IETIIGTHRSPVLTYHQACELLERTTSCGPFVQGLKGDYWDEEQQQYTG
VLANHLEQAWDKANKGIAPRNAYKLALKDELRPIEKNKAGKRRLLWGCD
AATTLIATAAFKAVATRLQVVTPMTPVAVGINMDSVQMQVMNDSLKGGV
LYCLDYSKWDSTQNPAVTAASLAILERFAEPHPIVSCAIEALSSPAEGY
VNDIKFVTRGGLPSGMPFTSVVNSINHMIYVAAAILQAYESHNVPYTGN
VFQVETVHTYGDDCMYSVCPATASIFHAVLANLTSYGLKPTAADKSDAI
KPTNTPVFLKRTFTQTPHGVRALLDITSITRQFYWLKANRTSDPSSPPA
FDRQARSAQLENALAYASQHGPVVFDTVRQIAIKTAQGEGLVLVNTNYD
QALATYNAWFIGGTVPDPVGHTEGTHKIVFEME

Preferably, the norovirus RdRp has an amino acid sequence according to SEQ NO: 4, SEQ ID NO: 5 or SEQ ID NO: 6:

SEQ ID NO: 4:
MGGDSKGTYCGAPILGPGSAPKLSTKTKFWRSSTTPLPPGTYEPAYLGGK
DPRVKGGPSLQQVMRDQLKPFTEPRGKPPKPSVLEAAKKTIINVLEQTID
PPEKWSFTQACASLDKTTSSGHPHHMRKNDCWNGESFTGKLADQASKANL
MFEGGKNMTPVYTGALKDELVKTDKIYGKIKKRLLWGSDLATMIRCARAF
GGLMDELKAHCVTLPIRVGMNMNEDGPIIFERHSRYKYHYDADYSRWDST
QQRAVLAAALEIMVKFSSEPHLAQVVAEDLLSPSVVDVGDFKISINEGLP
SGVPCTSQWNSIAHWLLTLCALSEVTNLSPDIIQANSLFSFYGDDEIVST
DIKLDPEKLTAKLKEYGLKPTRPDKTEGPLVISEDLNGLTFLRRTVTRDP
AGWFGKLEQSSILRQMYWTRGPNHEDPSETMIPHSQRPIQLMSLLGEAAL
HGPAFYSKISKLVIAELKEGGMDFYVPRQEPMFRWMRFSDLSTWEGDRNL
APSFVNEDGVEVDKLAAALE
SEQ ID NO: 5:
MGGDSKGTYCGAPILGPGSAPKLSTKTKFWRSSTTPLPPGTYEPAYLGGK
DPRVKGGPSLQQVMRDQLKPFTEPRGKPPKPSVLEAAKKTIINVLEQTID
PPEKWSFTQACASLDKTTSSGHPHHMRKNDCWNGESFTGKLADQASKANL
MFEGGKNMTPVYTGALKDELVKTDKIYGKIKKRLLWGSDLATMIRCARAF
GGLMDELKAHCVTLPIRVGMNMNEDGPIIFERHSRYKYHYDADYSRWDST
QQRAVLAAALEIMVKFSSEPHLAQVVAEDLLSPSVVDVGDFKISINEGLP
SGVPCTSQWNSIAHWLLTLCALSEVTNLSPDIIQANSLFSFYGDDEIVST
DIKLDPEKLTAKLKEYGLKPTRPDKTEGPLVISEDLNGLTFLRRTVTRDP
AGWFGKLEQSSILRQMYWTRGPNHEDPSETMIPHSQRPIQLMSLLGEAAL
HGPAFYSKISKLVIAELKEGGMDFYVPRQEPMFRWMRFSDLSTWEGDRNL
APSFVNEDGVEVDKLAAALEHHHHHH
SEQ ID NO: 6:
MHHHHHHGGDSKGTYCGAPILGPGSAPKLSTKTKFWRSSTTPLPPGTYEP
AYLGGKDPRVKGGPSLQQVMRDQLKPFTEPRGKPPKPSVLEAAKKTIINV
LEQTIDPPEKWSFTQACASLDKTTSSGHPHHMRKNDCWNGESFTGKLADQ
ASKANLMFEGGKNMTPVYTGALKDELVKTDKIYGKIKKRLLWGSDLATMI
RCARAFGGLMDELKAHCVTLPIRVGMNMNEDGPIIFERHSRYKYHYDADY
SRWDSTQQRAVLAAALEIMVKFSSEPHLAQVVAEDLLSPSVVDVGDFKIS
INEGLPSGVPCTSQWNSIAHWLLTLCALSEVTNLSPDIIQANSLFSFYGD
DEIVSTDIKLDPEKLTAKLKEYGLKPTRPDKTEGPLVISEDLNGLTFLRR
TVTRDPAGWFGKLEQSSILRQMYWTRGPNHEDPSETMIPHSQRPIQLMSL
LGEAALHGPAFYSKISKLVIAELKEGGMDFYVPRQEPMFRWMRFSDLSTW
EGDRNLAPSFVNEDGVEVDKLAAALE

The method of the present invention is suited to provide amplified RNA of all kinds and lengths. The method is particularly useful for providing short RNA molecules for gene silencing applications, either by antisense technology or RNA interference.

Therefore, the ssRNA template to be used in the method of the present invention may have short lengths of, e.g., 8 to 45 nucleotides, preferably of 15 to 30 nucleotides, preferably of 21 to 28 nucleotides, more preferably of 21 to 23 nucleotides. RNA molecules of the latter length are particularly useful for siRNA applications. In the case of amplifying short ssRNA templates, no primer or a short oligonucleotide for intiation of RNA-synthesis (oligoprimer) of e.g. 5 to 10 nucleotides may be used in the method of the present invention.

For de novo initiation of RNA synthesis (i.e. in the absence of a primer) it is preferred that the template contains at least 1, more preferred 1, 2, 3, 4 or 5, in particular 1 to 3 C nucleotides at its 3′ end.

Alternatively, the method of the present invention is also useful to provide longer RNA molecules, i.e. the ssRNA template has more than 30 or 45 nucleotides. A preferred embodiment of the inventive method makes use of mRNA templates.

The oligoprimer which may be optionally present may be selected as disclosed in WO 2007/12329 A2. Thus, by employing the method of the present invention, it is possible to select specific sequences of a ssRNA template by choosing (an) appropriate sequence-specific RNA-synthesis initiating oligonucleotide(s). Other possibilities include amplification of total mRNA from total cellular RNA by using a poly(U) RNA-synthesis initiating oligonucleotide.

According to the present invention, the terms “primer”, “oligoprimer” and “RNA-synthesis initiating oligonucleotide” are used interchangeably and refer to a short single-stranded RNA or DNA oligonucleotide capable of hybridizing to a target ssRNA molecule under hybridization conditions such that the sapovirus or norovirus RdRp is able to elongate said primer or RNA-synthesis oligonucleotide, respectively, under RNA polymerization conditions. In contrast to other RNA-dependent RNA polymerases, e.g. RNA-dependent RNA polymerases such as replicases of the Qβ type, the RNA polymerases of the caliciviruses useful in the present invention do not require primers having a specific recognition sequence for the polymerase to start RNA synthesis. Thus, a “primer”, oligoprimer” or “RNA-synthesis initiating oligonucleotide” as used herein is typically a primer not having such recognition sequences, in particular, of RNA polymerases. Furthermore, the calicivirus RNA polymerases to be used in the present invention are different from usual DNA-dependent RNA polymerases such as T7 RNA polymerase in that they do not require specific promoter sequences to be present in the template.

Furthermore, the method of the present invention is also useful to provide modified RNA molecules, in particular in the context of siRNA production. Thus, it is envisaged to include at least one labelled and/or modified nucleotide such as labelled and/or modified rNTPs or NTPs (e.g. 2′- or 3′-deoxy-modified nucleotides) in step (a) as defined above.

Chemically modified RNA products of the method of the present invention preferably have an increased stability as compared to the non-modified dsRNA analogues.

Especially for this purpose, the chemical modification of the at least one modified ribonucleoside triphosphate to be incorporated by the RdRp activity into the complementary strand can have a chemical modification(s) at the ribose, phosphate and/or base moiety. With respect to molecules having an increased stability, especially with respect to RNA degrading enzymes, modifications at the backbone, i.e. the ribose and/or phosphate moieties, are especially preferred.

Preferred examples of ribose-modified ribonucleoside triphosphates are analogues wherein the 2′-OH group is replaced by a group selected from H, OR, R, halo, SH, SR, NH₂, NHR, NR₂ or CN with R being C₁-C₆ alkyl, alkenyl or alkynyl and halo being F, Cl, Br or I. It is clear in the context of the present invention, that the term “modified ribonucleoside triphosphate” or “modified ribonucleotide” also includes 2′- or 3′-deoxy derivatives which may at several instances also be termed “deoxynucleotides”.

Typical examples of such ribonucleotide analogues with a modified ribose at the 2′ position include 2′-O-methyl-cytidine-5′-triphosphate, 2′-amino-2′-deoxy-uridine, 2′-azido-2′-deoxy-uridine-5′-triphosphate, 2′-fluoro-2′-deoxy-guanosine-5′-triphosphate and 2′-O-methyl-5-methyl-uridine-5′-triphosphate. For further details with regard to providing chemically modified RNA species by using the method of the present invention it is referred to co-pending International Patent Application No. PCT/EP2009/057119 (published as WO-A-2009/150156).

According to the present invention, it is not only possible to heat denature the produced dsRNA without the need to add further RdRp in each amplification cycle: the sapovirus and norovirus RdRps do not only withstand elevated temperatures such as 85° C., but these enzymes are also active at such elevated temperatures. Thus, the incubation step (a) can be carried out at a broad temperature range of, e.g. from 28 to 85° C. Elevated temperatures in step (a), e.g. 50 to 75° C. such as 60 to 65° C., are especially useful for amplifying RNA templates having secondary structures.

According to preferred embodiments of the present invention, microwave radiation may be used for carrying out the incubation steps (step (a) and, optionally step (d) and/or the separation step (b). Thus, the reaction composition present in the respective step(s) of the method according to the present invention is exposed to an amount of microwave radiation effective and sufficient to reach and maintain the reaction conditions as defined herein.

The term “effective amount of microwave energy” is the amount of microwave energy required for reaching and maintaining the desired temperature in the respective step(s) of the method according to the invention. The concrete amount of microwave energy for a given template may be determined by the skilled person using routine experimentation and depends particularly on the required temperature. For the polymerisation steps (step (a) and, optionally (d)), the microwave energy for reaching and maintaining the required reaction temperature (e.g. 28 to 65° C.) may be lower compared to the temperature in the separation step (b) (e.g. up to 85° C.). As used herein the terms “microwave energy”, “microwave (ir)radiation” or “irradiation with microwaves” or simply “microwaves” are used synonymously and relate to the part of the electromagnetic spectrum comprising wavelengths of about 0.3 to 30 cm, corresponding to a frequency of 1 to 100 gigahertz, which is found between the radio and the infra-red regions of the electromagnetic spectrum. The amount of electromagnetic energy absorbed by a living organism is determined by the dielectric properties of the tissues, cells, and biological molecules.

The generation of the microwave energy for the purposes of the present invention is not critical and can be by any means known to the art. For example, suitable means for applying microwave radiation to reaction compositions according to the invention are microwave ovens which are commercially available from numerous suppliers and routinely form part of the standard equipment in most biological laboratories. Such microwave ovens typically have maximum power levels of from about 500 W to about 1000 W. Even the smallest ovens provide ample levels of microwave irradiation for use in this invention and accordingly, it will be convenient to use lower power settings on ovens in which the output power is adjustable. Thus, according to preferred embodiments of the inventive methods disclosed herein, the composition is irradiated with microwaves having a frequency of from about 1500 MHz to about 3500 MHz and having a power of from about 50 to about 1000 W.

According to other embodiments of this invention, lower power settings are also used to time-distribute the applied power over a longer time interval and minimize the potential for localized energy uptake and resulting molecular damage. In an especially preferred embodiment, microwave power is applied to the sample over a series of intervals, with “rest” intervals, in which microwave power is not applied to the sample. Power application intervals and rest intervals will usually range from 1 to 60 seconds each, with power application intervals of from 15 to 60 seconds and rest intervals from 0.5 to 5 seconds being preferred. Most preferably, power will be applied for intervals of about 45 seconds, separated by rest intervals of 1 to 2 seconds.

However, especially depending on the length of the single-stranded polynucleotide template, the irradiation step may be carried out in a single application (interval) of microwave energy of a time period of 1 s to 5 min, more preferably 3 s to 120 s. The latter short time periods are especially useful when templates of shorter length (such as templates for preparing short dsRNAs such as siRNAs) are employed.

The figures show:

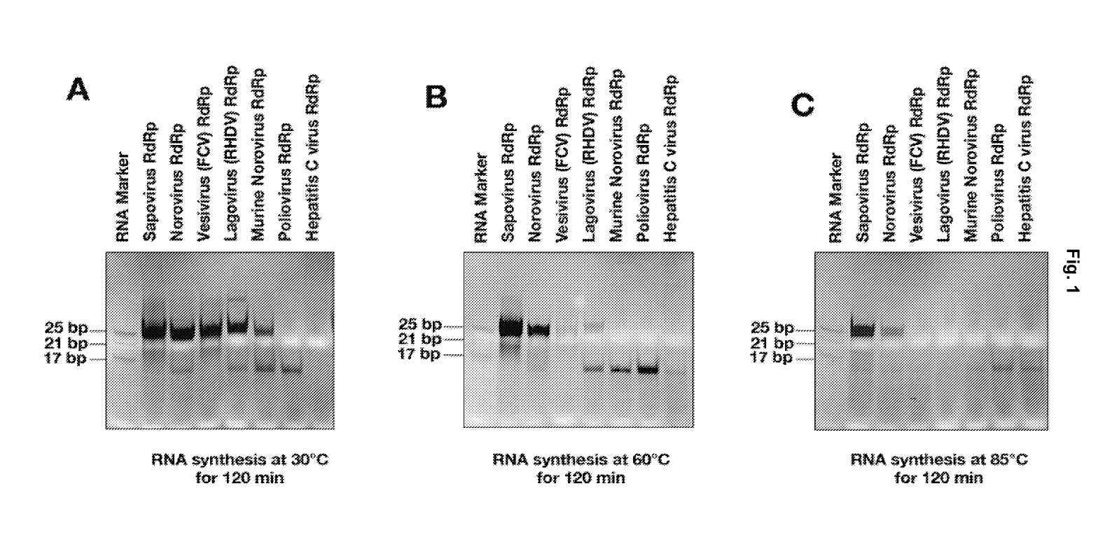

FIG. 1 shows photographs of native 20% polyacrylamide gels after electrophoresis of products of RNA synthesis on a single-stranded RNA template at different temperatures by RNA-dependent RNA polymerases as indicated. (A) Products of RNA synthesis at 30° C. for 2 h (120 min). (B) Products of RNA synthesis at 60° C. for 2 h (120 min). (C) Products of RNA synthesis at 85° C. for 2 h (120 min). The expected dsRNA product has a length of 24 bp. RNA Marker: dsRNA of 17 bp, 21 by and 25 bp.

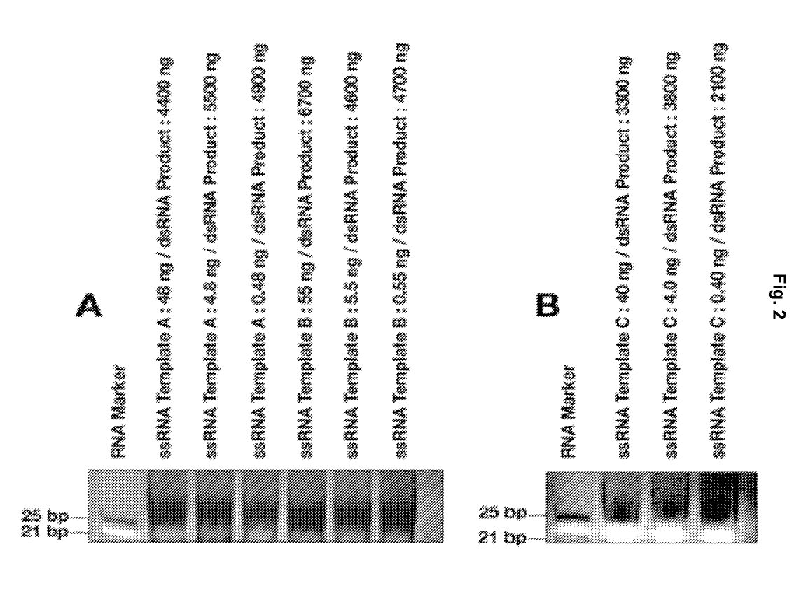

FIG. 2 shows photographs of native 20% polyacrylamide gels after electrophoresis of products of exponential RNA amplification by sapovirus RdRp on different ssRNA templates and various amounts thereof as indicated. The amplification was performed in 10 cycles of polymerization at 30° C. and denaturation at 85° C. (A) Analysis of amplification reaction using template A (23 nt) or template B (23 nt) in decreasing amounts per reaction as indicated. (B) Analysis of amplification reaction using template C (25 nt) in decreasing amounts per reaction as indicated. The amount of dsRNA product is indicated for each reaction.

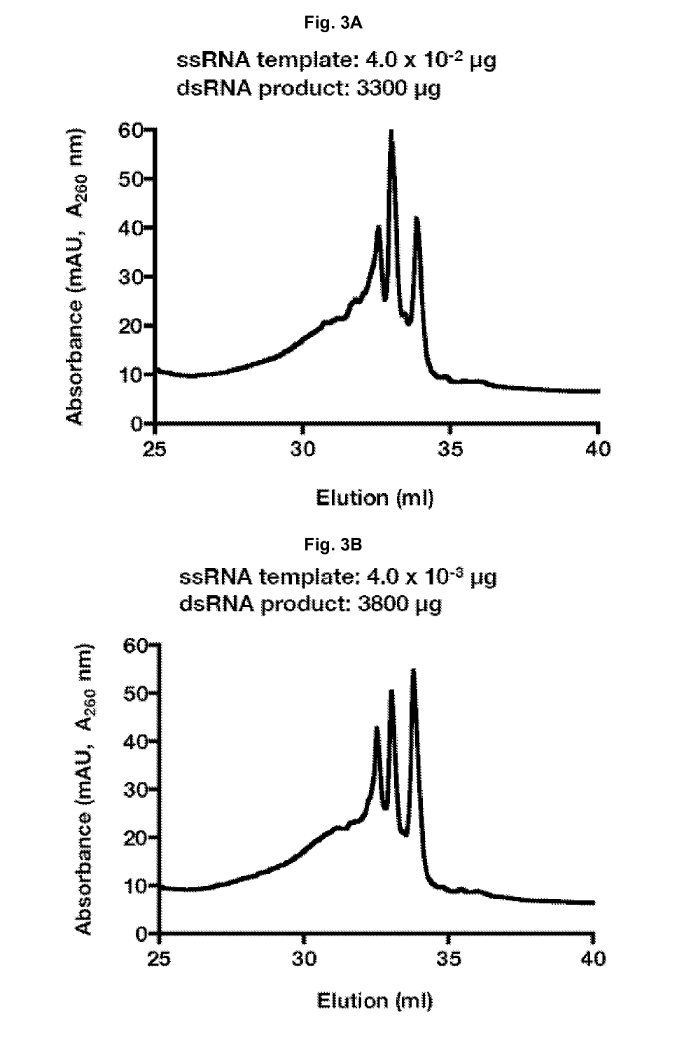

FIG. 3 shows graphical representations of elution profiles of ion exchange chromatographic analyses of double-stranded RNA products obtained by exponential amplification of single-stranded RNA by sapovirus RdRp. (A), (B), (C) Elution profiles of the dsRNA product resulting from template C (25 nt). The starting amount of the ssRNA template and the resulting amount of dsRNA product are indicated. (D) Superposition of elution profiles (A), (B) and (C).

The present invention is further illustrated by the following non-limiting examples.

### EXAMPLES

### Example 1

### The Sapovirus and Norovirus RdRp are Thermostable and Active at 85° C.

RNA synthesis was performed on a single-stranded RNA template of arbitrary sequence (24 nt) using the RNA-dependent RNA polymerase (RdRp) of the following viruses: sapovirus, genus Sapovirus, Family Calicivirdae; Norovirus, genus Norovirus, Family Calicivirdae; Feline calicivirus (FCV), genus Vesivirus, Family Calicivirdae; Rabbit Haemorrhagic disease virus (RHDV), genus Lagovirus, Family Calicivirdae; Murine Norovirus (MNV), genus Norovirus, Family Calicivirdae; Poliovirus, genus Enterovirus, Family Picornaviridae, and Hepatitis C virus, genus Hepacivirus, Family Flaviviridae. The reaction mix contained 1.5 μg of the template, 7.5 μM RdRp, 0.4 mM of each of rATP, rCTP, rUTP, and 2 mM rGTP, 10 μl reaction buffer (HEPES 250 mM, MnCl₂ 25 mM, DTT 5 mM, pH 7.6), and RNAse-DNAse free water to a total volume of 50 μl. The reaction was performed for 120 min (2 h) at 30° C., 60° C. or 85° C. The products were visualized on a native 20% polyacrylamide gel by electrophoresis (FIGS. 1A, 1B and 1C).

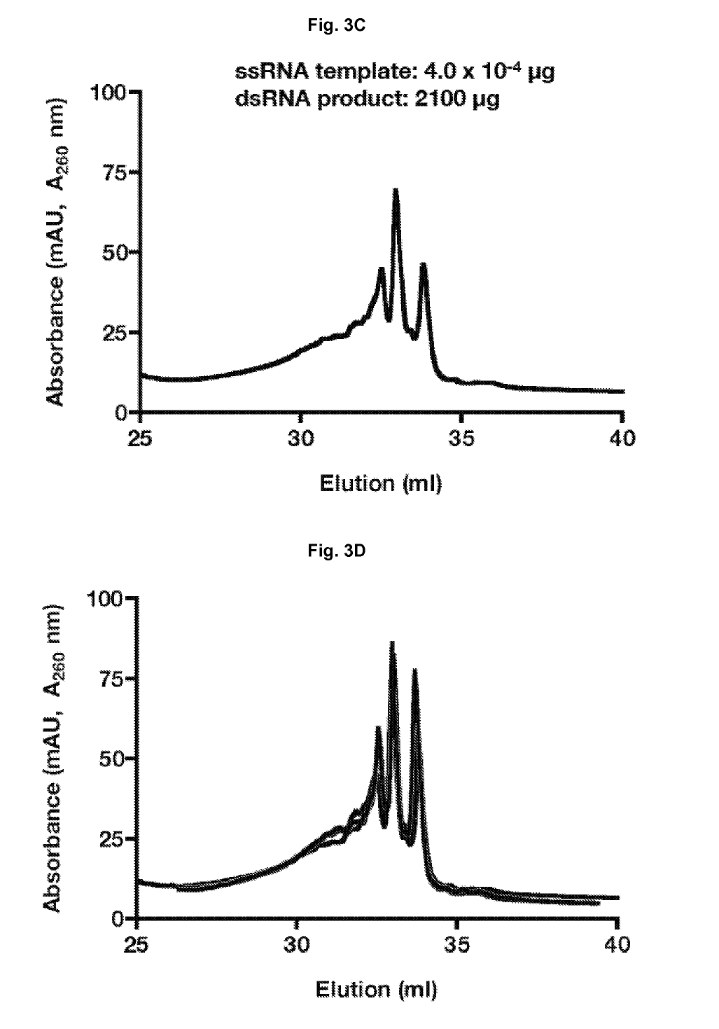

Primer-independent RNA synthesis was confirmed at 30° C. for all RdRps of the Caliciviridae family (FIG. 1A). At 60° C., the sapovirus and norovirus RdRps remained essentially active (FIG. 1B). Only weak product bands were obtained with the vesivirus and lagovirus RdRps at this temperature. At 85° C., the sapovirus RdRp generated a strong product band of 24 by (FIG. 10). A product band was also obtained with the norovirus RdRp at 85° C.

### Example 2

### Exponential Amplification of Single-Stranded RNA by the Sapovirus RdRp

RNA synthesis was performed on a single-stranded RNA template using the RNA-dependent RNA polymerase (RdRp) of the sapovirus. Three different templates named A (23 nt), B (23 nt) and C (25 nt) were used in different amounts. The reaction mix contained three different amounts of each template (template A: 48 ng, 4.8 ng, 0.48 ng; template B: 55 ng, 5.5 ng, 0.55 ng; template C: 40 ng, 4.0 ng, 0.40 ng). 7.5 μM RdRp, 1.2 mM of each of rATP, rCTP, rUTP, and 6 mM rGTP, 30 μl reaction buffer (HEPES 250 mM, MnCl₂ 25 mM, DTT 5 mM, pH 7.6), and RNAse-DNAse free water to a total volume of 150 μl. The amplification reaction was performed in 10 successive cycles, each cycle consisting of incubation at 30° C. for 15 min, followed by denaturation at 85° C. for 5 minutes. The products were visualized on a native 20% polyacrylamide gel by electrophoresis (FIGS. 2A and 2B).

The reactions resulted in dsRNA in the amounts indicated in FIGS. 2A and 2B, respectively. The amount of dsRNA synthesised was determined by using the RiboGreen fluorescent dye (Invitrogen) measured on the TECAN Infinite 200.

The results of the product measurements are summarised in the following Table 1:

TABLE 1
Analysis of amount of dsRNA products

| | Amount of ssRNA | Amount of dsRNA | Amplification |
|---|---|---|---|
| Template | template [μg] | product [μg] | [fold] |
| A | 4.8 × 10⁻² | 4.4 | 93 |
| A | 4.8 × 10⁻³ | 5.5 | 1145 |
| A | 4.8 × 10⁻⁴ | 4.9 | 12250 |
| B | 5.5 × 10⁻² | 6.7 | 121 |
| B | 5.5 × 10⁻³ | 4.6 | 832 |
| B | 5.5 × 10⁻⁴ | 4.7 | 8509 |
| C | 4.0 × 10⁻² | 3.3 | 83 |
| C | 4.0 × 10⁻³ | 3.8 | 937 |
| C | 4.0 × 10⁻⁴ | 2.1 | 5250 |

Having in mind the drawbacks of prior art RNA amplification methods (see the prior art mentioned above), it is remarkable that the RNA amplification reaction according to the present invention is highly efficient even as compared to established PCR protocols: whereas PCR protocols typically result in acceptable amounts of product DNA after 40 cycles, the RNA amplification protocol of the present invention results in a more than 10,000 fold amplification after 10 cycles only.

### Example 3

### Chromatographic Analysis of dsRNA Product Resulting from Exponential Amplification of ssRNA by the Sapovirus RdRp

The amplification reactions obtained with ssRNA template C as described in Example 2 were resolved on a DNAPak PA100 (Dionex) ion exchange column. Almost identical elution profiles were obtained for all three reactions (FIGS. 3A, 3B and 3D) as confirmed by superposition of the elution profiles (FIG. 3D).

What is claimed is:

1. A method for exponential amplification of RNA in vitro comprising the steps of:
(a) incubating single-stranded RNA (ssRNA) with a RNA-dependent RNA polymerase (RdRp) of a sapovirus or norovirus, optionally in the presence of a RNA-synthesis initiating oligonucleotide (oligoprimer), under conditions such that the RdRp polymerizes a strand complementary to the ssRNA, optionally by elongating said oligoprimer hybridized to said ssRNA, to form double-stranded RNA (dsRNA);
(b) incubating the reaction mixture obtained in step (b) at a temperature of at most 85° C. such that the duplex of the dsRNA is separated into ssRNA;
(c) repeating steps (a) and (b) n times;
(d) performing a final incubation step (a) to form final dsRNA; and, optionally,
(e) recovering the final dsRNA;
wherein
n is at least 3;
and the sequence and/or length of the ssRNA is selected such that the dsRNA formed in step (b) is separated into ssRNA at a temperature of at most 85° C.

2. The method of claim 1 wherein n≧5 and further RdRp is added between steps (b) and (a) at every 3^{rd} to 5^{th} cycle of step (c).

3. The method of claim 1 wherein the temperature in step (b) is from 65° C. to 85° C.

4. The method of claim 1 wherein n is 5 to 40.

5. The method of claim 4 wherein n is 20.

6. The method of claim 1 wherein the incubation in step (a) is carried out at a temperature of from 28 to 85° C.

7. The method of claim 6 wherein the temperature is from 50 to 75, preferably 60° C. to 65° C.

8. The method of claim 1 wherein the sapovirus RdRp is an RdRp of the sapovirus strain pJG-Sap01 (GenBank Acc. No. AY694184).

9. The method of claim 8 wherein the RdRp has an amino acid sequence selected from the group consisting of SEQ ID NO: 1, SEQ ID NO: 2 and SEQ ID NO: 3.

10. The method of claim 1 wherein the norovirus RdRp is an RdRp of the norovirus strain NuCV/NL/Dresden174/1997/GE (GenBank Acc. No. AY741811).

11. The method of claim 10 wherein the RdRp has an amino acid sequence selected from the group consisting of SEQ ID NO: 4, SEQ ID NO: 5 and SEQ ID NO: 6.

12. The method of claim 1 wherein the ssRNA template has a length of from 15 to 30, preferably 21 to 28 nucleotides, more preferably 21 to 23 nucleotides.

13. The method of claim 1 wherein the ssRNA template has a length of more than 30 nucleotides.

14. The method of claim 13 wherein the ssRNA template is mRNA.

15. The method of claim 1 wherein at least one modified and/or labelled nucleotide is present in step (a).

16. The method of claim 1 wherein the incubation step(s) (a), and optionally (d) and/or the separation step (b) is/are carried out under microwave irradiation.


SEQUENCE LISTING (6 sequences)

SEQ ID NO: 1 - protein, 517 aa, Sapovirus
MKDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEME

SEQ ID NO: 2 - protein, 526 aa, Artificial
  Sapovirus-RdRp having C-terminal His-tag
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALEHHHHHH

SEQ ID NO: 3 - protein, 523 aa, Artificial
  Sapovirus-RdRp having N-terminal His-tag
MKHHHHHHDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEME

SEQ ID NO: 4 - protein, 520 aa, Norovirus
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALE

SEQ ID NO: 5 - protein, 526 aa, Artificial; identical to SEQ ID NO: 2
  Norovirus-RdRp having C-terminal his tag

SEQ ID NO: 6 - protein, 456 aa, Artificial
  Norovirus-RdRp having N-terminal His-tag
MHHHHHHGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAAL